(19) United States
(12) Patent Application Publication
(10) Pub. No.: US 2019/0374169 A1
(43) Pub. Date: Dec. 12, 2019

(54) INFORMATION PROCESSING APPARATUS AND INFORMATION PROCESSING PROGRAM

(71) Applicants: OMRON CORPORATION, Kyoto-shi (JP); OMRON HEALTHCARE CO., LTD., Muko-shi (JP)

(72) Inventors: Kosuke INOUE, Kyoto (JP); Shusuke ESHITA, Kyoto (JP)

(73) Assignees: OMRON CORPORATION, Kyoto-shi (JP); OMRON HEALTHCARE CO., LTD., Muko-shi (JP)

(21) Appl. No.: 16/548,994

(22) Filed: Aug. 23, 2019

Related U.S. Application Data
(63) Continuation of application No. PCT/JP2018/004916, filed on Feb. 13, 2018.

(30) Foreign Application Priority Data
Mar. 14, 2017 (JP) 2017-048737

(51) Int. Cl. A61B 5/00; A61B 5/0205

(52) U.S. Cl. CPC A61B 5/7275; A61B 5/0205; A61B 5/4818; A61B 5/021; A61B 5/743; A61B 5/0002; A61B 5/7246

(57) ABSTRACT

An information processing apparatus of the invention includes an information acquisition circuit configured to acquire, from a specific subject to be measured, measurement data of a successively measured blood pressure value and measurement data of SPO2, a blood pressure fluctuation detection circuit configured to detect a blood pressure fluctuation of a reference value or more from the measurement data of the successively measured blood pressure value acquired by the information acquisition circuit, and a correlation determination circuit configured to determine a degree of possibility of occurrence of a blood pressure fluctuation by sleep apnea syndrome.

### CROSS-REFERENCE TO RELATED APPLICATIONS

This application is a Continuation Application of PCT Application No. PCT/JP2018/004916, filed Feb. 13, 2018 and based upon and claiming the benefit of priority from Japanese Patent Application No. 2017-048737, filed Mar. 14, 2017, the entire contents of all of which are incorporated herein by reference.

### FIELD

The present invention relates to an information processing apparatus and an information processing program, and more specifically to an information processing apparatus and an information processing program, which display, on a display screen, information relating to a blood pressure of a subject to be measured.

### BACKGROUND

Conventionally, as an apparatus which displays information relating to a blood pressure of a subject to be measured, for example, as disclosed in Jpn. Pat. Appln. KOKAI Publication No. 2004-261452, there is proposed a blood pressure monitor which stores in a memory a measured blood pressure value, and measurement time information and a measurement condition by mutually associating the measured blood pressure value, and the measurement time information and measurement condition, calculates an average value which is acquired by averaging blood pressure values measured multiple times in a specified time slot, and calculates and displays a risk value, based on the calculated result.

In addition, in recent years, it is proposed that a blood pressure of a subject to be measured is successively measured for a long time (e.g. at night, or 24 hours) by a portable blood pressure monitor, and healthcare of the subject to be measured is performed based on the measured result.

### SUMMARY

An information processing apparatus according to a first aspect of the invention comprises an information acquisition circuit configured to acquire, from a specific subject to be measured, measurement data of a successively measured blood pressure value and measurement data of SPO2, a blood pressure fluctuation detection circuit configured to detect a blood pressure fluctuation of a reference value or more from the measurement data of the successively measured blood pressure value acquired by the information acquisition circuit, a correlation determination circuit configured to determine a degree of possibility of occurrence of a blood pressure fluctuation by sleep apnea syndrome, based on a correlation between the blood pressure fluctuation of the reference value or more, which the blood pressure fluctuation detection circuit detects, and the measurement data of the SPO2, and a display controller configured to cause a display device to display information indicative of the degree of the possibility of the occurrence of the blood pressure fluctuation by the sleep apnea syndrome, which the correlation determination circuit determines.

In addition to the first aspect, in the information processing apparatus according to a second aspect of the invention, the display controller is configured to cause the display device to further display advice on medical treatment corresponding to the degree of the possibility of the occurrence of the blood pressure fluctuation by the sleep apnea syndrome, which the correlation determination circuit determines.

In addition to the first aspect, the information processing apparatus according to a third aspect of the invention further comprises a comparative analysis circuit configured to acquire, when an instruction for comparison with past measurement data is given, past measurement data relating to the subject to be measured, who is being displayed, and configured to generate a comparison screen which compares measurement data, which is being displayed, and the past measurement data, wherein the display controller is configured to cause the display device to display the comparison screen which the comparative analysis circuit generates in accordance with the instruction for the comparison with the past measurement data.

In addition to the third aspect, in the information processing apparatus according to a fourth aspect of the invention, the comparative analysis circuit is configured to create a comment based on the comparison between the measurement data, which is being displayed, and the past measurement data, and the display controller is configured to cause the display device to further display the comment which the comparative analysis circuit creates, together with the comparison screen.

In addition to any one of the first to fourth aspects, the information processing apparatus according to a fifth aspect of the invention further comprises a correlation information generation circuit configured to generate a correlation graph which indicates a correlation between the measurement data of the SPO2 acquired by the information acquisition circuit and the blood pressure fluctuation of the reference value or more, which the blood pressure fluctuation detection circuit detects, wherein the display controller is configured to cause the display device to display the correlation graph which the correlation information generation circuit generates in accordance with display instruction of an operator.

In addition to any one of the first to fifth aspects, in the information processing apparatus according to a sixth aspect of the invention, the information acquisition circuit is configured to acquire successively measured blood pressure data by a blood pressure sensor of any one of a PTT method, a tonometry method, an optical method, a radio wave method, and ultrasonic method.

According to the first aspect of the present invention, the degree of possibility of the occurrence of a blood pressure fluctuation by sleep apnea syndrome, which SPO2 indicates, can be determined based on the measurement data of the blood pressure value which is successively measured, and the measurement data of SPO2, and the determination result can be displayed. As a result, the user or medical staff can easily understand the degree of possibility of the occurrence of a blood pressure fluctuation by sleep apnea syndrome with respect to each individual.

According to the second aspect of the present invention, the advice on medical treatment corresponding to the degree of possibility of the occurrence of a blood pressure fluctuation by sleep apnea syndrome can be displayed, and it is possible to support a medical treatment and an improvement of a lifestyle, which correspond to the degree of possibility of the occurrence of a blood pressure fluctuation by sleep apnea syndrome.

According to the third aspect of the present invention, the comparison screen with past measurement data can be displayed, and the past measurement data and present measurement data can be viewed by comparison. Thereby, a medical treatment and an improvement of a lifestyle, which correspond to the details in the past, can be supported.

According to the fourth aspect of the present invention, a comment based on the comparison with past measurement data can be displayed, and, based on the comment, a medical treatment and an improvement of a lifestyle, which correspond to the details in the past, can be supported.

According to the fifth aspect of the present invention, a correlation graph indicative of the correlation between the measurement data of SPO2 and the blood pressure fluctuation of the reference value or more can be displayed, and a medical treatment and an improvement of a lifestyle, which correspond to the correlation between SPO2 and the blood pressure fluctuation, can be supported.

According to the sixth aspect of the present invention, the successively measured blood pressure data is not limited to the data measured by the blood pressure sensor of a specific method. It is possible to provide information indicative of the degree of possibility of the occurrence of the blood pressure fluctuation by sleep apnea syndrome, based on the blood pressure data measured by measuring devices of various methods.

### BRIEF DESCRIPTION OF THE DRAWINGS

FIG. 1 is a view schematically illustrating a configuration example of a management system of measurement data, which includes an information processing system functioning as an information processing apparatus according to an embodiment of the present embodiment.

FIG. 2 is a block diagram illustrating a configuration example of a measuring terminal illustrated in FIG. 1.

FIG. 3 is a block diagram illustrating a configuration example of a user terminal illustrated in FIG. 1.

FIG. 4 is a block diagram illustrating a configuration example of a server illustrated in FIG. 1.

FIG. 5 is a block diagram illustrating a configuration example of a medical staff terminal illustrated in FIG. 1.

FIG. 6 is a block diagram for explaining functions which the server illustrated in FIG. 1 includes.

FIG. 7 is a view illustrating a transition example of an operation screen which is displayed when measurement is performed in the measuring terminal illustrated in FIG. 1.

FIG. 8 is a view illustrating a display example of blood pressure-related information which the information processing system displays.

FIG. 9 is a view illustrating a display example of a graph indicative of a correlation between measurement data of SPO2 and a blood pressure surge, which the information processing system displays.

FIG. 10 is a view illustrating a display example of blood pressure-related information of a person having a history of medical treatment, which the information processing system displays.

FIG. 11 is a view illustrating a display example of a comparison screen for comparison with past measurement data, which the information processing system displays.

FIG. 12 is a flowchart for explaining an operation example of the server functioning as the information. processing system.

FIG. 13 is a flowchart for explaining an operation example of the server functioning as the information processing system.

### DETAILED DESCRIPTION

Hereinafter, embodiments according to the present invention will be described with reference to the accompanying drawing.

It is proposed that a blood pressure of a subject to be measured is successively measured for a long time (e.g. at night, or 24 hours) by a portable blood pressure monitor, and healthcare of the subject to be measured is performed based on the measured result.

However, even if an enormous amount of data of blood pressure values acquired by successive measurement over a long time is simply presented to a user, it is difficult for the user to correctly understand the health condition. In addition, even for a person (hereinafter referred to as “medical staff”) such as a doctor, who has expertise in the field of medical practice and health, it is a great load for the person to understand the health condition of the subject to be measured, from the enormous amount of data of blood pressure values.

Besides, there are various conceivable factors in a sharp increase (surge) of a blood pressure, which is considered to have a close relation to an incidence risk of a cerebral cardiovascular event. For example, sleep apnea syndrome (SAS) is known as a possible factor in the blood pressure surge. However, since there is a variance among individuals due to a constitutional predisposition or the like, it varies from individual to individual how much the SAS is related to the blood pressure surge. It is a great load for medical staff to judge such a correlation between SAS and the blood pressure surge with respect to each individual. Thus, in order to prevent a blood pressure surge in which SAS becomes a factor, there is a demand for a technology which can support a proposal for a medical treatment or an improvement of lifestyle in accordance with the constitutional predisposition of each individual.

The embodiment of the present invention has been made in consideration of the above-described circumstance, and the object of the invention is to provide an information processing apparatus and an information processing program, which can make it possible to easily understand a blood pressure fluctuation due to sleep apnea syndrome for each individual.

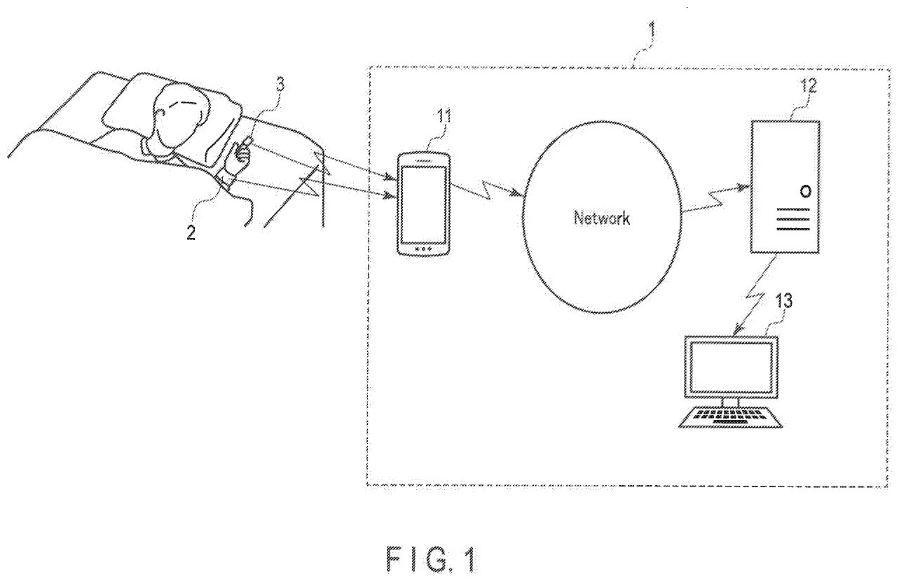

FIG. 1 is a view schematically illustrating a configuration example of a management system of measurement data according to an embodiment.

The management system of measurement data is a system including an information processing system 1 functioning as an information processing apparatus, a measuring terminal 2, and a sensor 3. The information processing system 1 acquires, from the measuring terminal 2 and sensor 3, measurement data including successively measured blood pressure data and measurement data of various elements, and analyzes the acquired measurement data. In addition, in the configuration example illustrated in FIG. 1, the information processing system 1 includes a user terminal 11, a server 12 and a medical staff terminal 13.

In the configuration example illustrated in FIG. 1, the measuring terminal 2 and sensor 3 are connected to the user terminal 11, and the user terminal 11 is communicably connected to the server 12 via a network. Further, the server 12 is communicably connected to the medical staff terminal 13 by wireless communication or wire communication. However, the configurations of the management system of measurement data and the information processing system 1 are not limited to the configurations illustrated in FIG. 1.

For example, the measuring terminal 2 and sensor 3 may be communicably connected to the server 12, without intervention of the user terminal 11. In this case, the user terminal 11 can be omitted, and the operation of the user terminal 11, which will be described later, may be implemented by the measuring terminal 2 or server 12. In addition, the sensor 3 may be connected to not the user terminal 11 but the measuring terminal 2. In this case, the sensor 3 may be configured to be communicable with the user terminal 11 or server 12 via the measuring terminal 2. Further, the sensor 3 may be provided in the measuring terminal 2.

Besides, some or all of the functions (processes), which the server 12 to be described later realizes, may be implemented by the user terminal 11 or medical staff terminal 13, or may be implemented by the cooperation between the user terminal 11 or medical staff terminal 13, and the server 12. Further, some or all of the functions of the user terminal 11 to be described later, the functions of the server 12 and the functions of the medical staff terminal 13 may be implemented by the measuring terminal 2. For example, the information which the server 12 provides may be displayed by a display unit of the user terminal 11 or may be displayed by a display unit of the measuring terminal 2.

The measuring terminal 2 includes a function of successively measuring at least the blood pressure value of a subject to be measured (user). The measuring terminal 2 is, for example, a wearable terminal device such as a wristwatch-type terminal device. The measuring terminal 2 includes not only the function of successively measuring the user's blood pressure values, but also a function of measuring biological data such as an activity amount, the number of steps and a sleep state, and environmental data such as a temperature and humidity.

The measuring terminal 2 includes a plurality of sensors for measuring the biological data and environmental data. For example, the measuring terminal 2 includes a biological sensor group (biological sensors) including sensors for detecting signals indicative of values of various kinds of biological information, such as a blood pressure, in a state of being in contact with or in close proximity to a part of the body of the subject to be measured. A biological sensor that the measuring terminal 2 includes is configured to be put in contact with, or in close proximity to, a predetermined position on the subject to be measured, for example, by a band or the like. In addition, the measuring terminal 2 also includes an environmental sensor group (environmental sensors) including sensors for detecting signals indicative of values of a temperature, humidity and the like, which represent the environment in which the subject to be measured is present.

The sensor 3 is a sensor which detects a signal indicative of a value of a specific element that is a target of measurement. In the present embodiment, it is assumed that the sensor 3 is a sensor which detects a value of arterial oxygen saturation (SPO2) indicative of the state of sleep apnea syndrome (SAS). The sensor 3 which detects the value of SPO2 is worn on, for example, a predetermined part (e.g. a fingertip) of the subject to be measured. The sensor 3 includes a communication function for communication with the user terminal 11, and sends measured data to the user terminal. The sensor 3 may be communicably connected to the measuring terminal 2, or may include a function of a communicable connection to the server 12. Note that in the system configuration illustrated in FIG. 1, the sensor 3 is the sensor which detects SPO2, but the management system of measurement data, as a whole, may include a plurality of kinds of sensors.

The user terminal 11 is an information communication terminal which an individual user uses. The user terminal 11 is, for example, a portable information communication terminal such as a smartphone, a mobile phone, a tablet PC, or a note PC. It should suffice if the user terminal 11 is capable of transferring at least data measured by the measuring terminal 2 and sensor 3 to the server 12. In the management system of measurement data, the user terminal 11 is present at least for each user. In addition, an individual user may have a plurality of user terminals.

The server 12 includes a communication function with the user terminal 11, and a communication function with the medical staff terminal 13. In the configuration example illustrated in FIG. 1, it is assumed that the server 12 communicates with the user terminal 11 via a wide area network, and communicates with the medical staff terminal 13 via a local area network. However, it should suffice if the server 12 can communicate with both the user terminal 11 and medical staff terminal 13, and the method of communication and the mode of communication are not limited to specific ones. The server 12 acquires measurement data, which is measured by the measuring terminal 2 and sensor 3, from the user terminal 11, and analyzes the acquired measurement data. In addition, the server 12 provides the measurement data, the analysis result of measurement data and the like to the medical staff terminal 13.

The medical staff terminal 13 an information communication apparatus which medical staff uses who instructs a treatment policy to the user or proposes an improvement of a lifestyle to the user. The user terminal 11 is, for example, an information communication apparatus including an information display function, such as a desktop PC, a note PC, or a tablet PC. The medical staff terminal 13 displays, on a display device, information which is provided from the server 12. In addition, the medical staff terminal 13 includes a function of requesting the server 12 to execute various processes (information display) in accordance with an operation by medical staff.

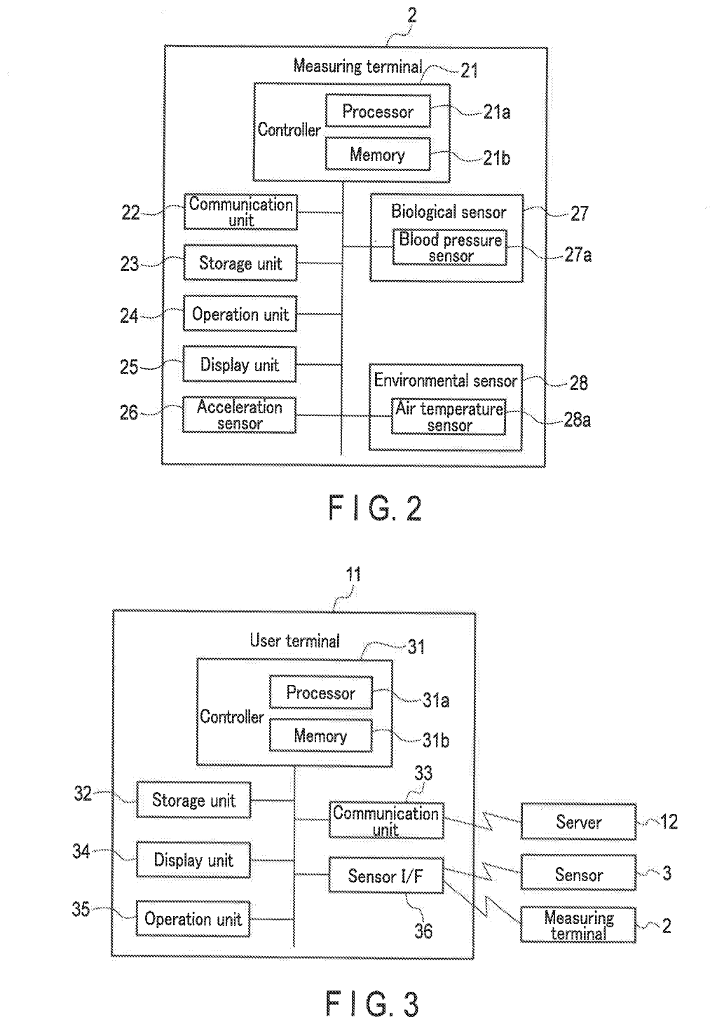

FIG. 2 is a block diagram illustrating a configuration example of the measuring terminal 2 illustrated in FIG. 1.

The measuring terminal 2 includes a controller 21, a communication unit 22, a storage unit 23, an operation unit 24, a display unit 25, an acceleration unit 26, a biological sensor 27, and an environmental sensor 28.

The controller 21 includes at least one processor 21a, and a memory 21b. The processor 21a executes programs by using the memory 21b, and thereby the controller 21 realizes various kinds of operation control and data processing. The processor 21a is, for example, a CPU (Central Processing Unit) or an MPU (Micro Processing Unit) including arithmetic circuitry. The memory 21b includes a nonvolatile memory (a non-transitory computer-readable storage medium) which stores programs that the processor 21a executes, and a memory such as a RAM which is used as a work memory. In addition, the controller 21 includes a clock (not shown) and includes a clock function which measures the present date/time.

In the controller 21, the processor 21a executes programs which the memory 21b or storage unit 23 (a non-transitory computer-readable storage medium) stores, and thereby the processor 21a can execute control of respective components and data processing. Specifically, the processor 21a executes operational control of respective components according to operation signals from the operation unit 24, and executes data processing on measurement data which the biological sensor 27 and environmental sensor 28 measure. For example, the controller 21 executes an operation in a mode (screening mode) which successively measures various kinds of information including a blood pressure value of a subject to be measured, in accordance with an instruction by the operation unit 24.

The communication unit 22 is a communication interface for communicating with the user terminal 11. The communication unit 22 transmits data to the user terminal 11, and receives data from the user terminal 11. The communication by the communication unit 22 may be either wireless communication or wire communication. In the present embodiment, the description is given on the assumption that the communication unit 22 communicates with the user terminal 11 by short-range wireless communication. However, aside from this, the communication unit 22 may communicate with the user terminal 11 by using a communication cable, or may communicate with the user terminal 11 via a network such as a LAN (Local Area Network).

The storage unit 23 stores data of programs for controlling the measuring terminal 2, setup data for setting up various functions of the measuring terminal 2, and measurement data which the acceleration sensor 26, biological sensor 27 and environmental sensor 28 measure. In addition, the storage unit 23 may be configured to be used as a work memory when a program is executed.

The operation unit 24 is composed of operation devices such as a touch panel and operation buttons (operation keys). The operation unit 24 detects an operation by a user (a subject to be measured), and outputs an operation signal indicative of the content of the operation to the controller 21. In addition, the operation unit 24 may include, for example, aside from the touch panel and operation buttons, a speech recognition unit which recognizes an operation instruction by the user's speech, a biological authentication unit which authenticates a biological part of the user, and an image recognition unit which recognizes the user's facial expression or gesture by an image acquired by photographing the user's face or body.

The display unit 25 includes, for example, a display screen (for example, an LCD (Liquid Crystal Display), EL (Electroluminescence) display or the like), an indicator, and the like, and displays information according to a control signal from the controller 21. In the present embodiment, the description is given on the assumption that the operation unit 24 and display unit 25 are composed of a display including a touch panel.

The acceleration sensor 26 detects an acceleration which the main body of the measuring terminal 2 receives. For example, the acceleration sensor acquires acceleration data of three axes or six axes. The acceleration data can be used in order to estimate the activity amount (posture and/or motion) of the user wearing the measuring terminal 2. For example, when the user is sleeping, a change in posture of the subject to be measured, which can be estimated from the acceleration data, may become data indicative of the sleep state (depth of sleep) of the subject to be measured. In this case, the controller 21 correlates the acceleration data, which the acceleration sensor 26 measures, with measurement time instants, and outputs the correlated data as measurement data in the sleep state. In addition, when the user is up, a change in motion, which can be estimated from the acceleration data, may become data indicative of the activity amount (e.g. activity amount by exercise such as walking or running) of the user. In this case, the controller 21 correlates the acceleration data, which the acceleration sensor 26 measures, with the measurement time instants, and outputs the correlated data as measurement data of the activity amount.

Note that whether the user is sleeping or up may be detected based on the user's motion which the acceleration sensor 26 detects, or whether the user is sleeping or up may be determined according to the user's operation. In the latter case, for example, the user may indicate that the user is in the sleep state before the user sleeps by the operation unit 24, or may indicate that the user is up at the time of getting up by the operation unit 24.

The biological sensor 27 measures biological information of the user, and outputs biological data that is measurement data of the biological information. The controller 21 acquires each measurement data, which the biological sensor 27 outputs, as biological data correlated with measurement time instants which are set based on time instant information. The biological sensor 27 includes at least a blood pressure sensor 27a. The blood pressure sensor 27a acquires blood pressure data as biological data in which the blood pressure value of the user is successively measured.

It is assumed that the biological data, which the biological sensor 27 acquires, are, aside from the blood pressure value, pulse wave data, pulse data, electrocardiographic data, heartbeat data and body temperature data. Sensors for measuring these biological data may be provided as the biological sensor 27. The biological data may be output as measurement data of elements other than the blood pressure. For example, brain wave data can be acquired as measurement data indicative of the sleep state of a human.

The blood pressure sensor 27a is a blood pressure sensor of a successive measurement type. The blood pressure sensor 27a is a blood pressure sensor which can successively measure the values of the blood pressure (e.g. a systolic blood pressure and a diastolic blood pressure). The blood pressure sensor 27a may include a blood. pressure sensor which can successively measure a blood pressure on a pulse-by-pulse basis, but the blood pressure sensor 27a is not limited to this.

For example, as the blood pressure sensor 27a, a successive measurement-type blood pressure sensor using a PTT method, tonometry method, optical method, radio wave method or ultrasonic method is applicable. The PTT method is a method of measuring a pulse transmit time (PTT), and estimating a blood pressure value from the measured PTT. The tonometry method is a method of putting a pressure sensor in direct contact with a biological part where an artery, such as a radial artery of the wrist, passes, and measuring a blood pressure value by using information which the pressure sensor detects. The optical method, radio wave method and ultrasonic method are methods of applying light, radio waves or ultrasonic waves to a blood vessel, and measuring a blood pressure value from the reflected waves.

Note that the blood pressure sensor of the successive measurement type can measure a blood pressure waveform of the user, can acquire blood pressure values, based on the measured blood pressure waveform, and can calculate a heart rate, based on the cycle of the measured blood pressure waveform. The heartbeat data may include, for example, a heart rate, but the heartbeat data is not limited to the heart rate. The heart rate may be measured by a heartbeat sensor, as well as by the successive measurement-type blood pressure sensor.

The environmental sensor 28 includes a sensor which measures environmental information of the surrounding of the user, and acquires measured environmental data. In the configuration example illustrated in FIG. 2, it is assumed that the environmental sensor 28 includes an air temperature sensor 28a. However, the environmental sensor 28 may include, as well as the air temperature sensor, sensors which measure a temperature, humidity, sound, light, and the like. It should suffice if the environmental sensor 28 includes a sensor which measures environmental information (environmental data) that is assumed to have a direct or indirect relation to a fluctuation of the blood pressure value. Further, the controller 21 correlates measurement data, which the environmental sensor 28 measures, with measurement time instants which are set based on time instant information, and acquires the correlated data as measurement data (environmental data).

Next, a configuration of the user terminal 11 will be described.

FIG. 3 is a block diagram illustrating a configuration example of the user terminal 11 illustrated in FIG. 1.

In the configuration example illustrated in FIG. 3, the user terminal 11 includes a controller 31, a storage unit 32, a communication unit 33, a display unit 34, an operation unit 35, and a sensor interface (I/F) 36. In the present embodiment, the user terminal 11 is, for example, a portable communication terminal such as a smartphone or a tablet. Application software (program) is installed in the user terminal 11 such that a process to be described later is executable.

The controller 31 includes at least one processor 31a, and a memory 31b. The processor 31a executes programs by using the memory 31b, and thereby the controller 31 realizes various kinds of operation control and data processing. The processor 31a is, for example, a CPU or an MPU including arithmetic circuitry. The memory 31b includes a nonvolatile memory (a non-transitory computer-readable storage medium) which stores programs that the processor 31a executes, and a volatile memory such as a RAM which is used as a work memory. In addition, the controller 31 includes a clock (not shown) and includes a clock function which measures the present date/time.

The storage unit 32 is a data memory. The storage unit 32 is composed of, for example, a semiconductor memory (a memory card, an SSD (Solid State Drive) or a magnetic disk (HD (Hard Disk)). The storage unit 32 may store programs which the processor 31a of the controller 31 executes. In addition, the storage unit 32 may store measurement data which is supplied from the measuring terminal 2 and sensor 3. Further, the storage unit 32 may also store display data which is displayed on the display unit.

The communication unit 33 is a communication interface for communicating with the server 12. The communication unit 33 transmits data to the server 12 via a network, and receives data from the server 12. The communication by the communication unit 33 may be either wireless communication or wire communication. In the present embodiment, the description is given on the assumption that the network is, for example, the Internet or the like. However, the network is not limited to this, and may be another kind of network such as a LAN, or the communication may be one-to-one communication using a communication cable such as a USB cable.

The display unit 34 includes a display screen (for example, an LCD, an EL display or the like). In the display unit 34, the display content, which is displayed on the display screen, is controlled by the control of the controller 31.

The operation unit 35 sends to the controller 31 an operation signal corresponding to an operation by the user (e.g. the subject to be measured). The operation unit 35 is, for example, a touch panel provided on the display screen of the display unit 34. The operation unit 35 is not limited to the touch panel, and may be an operation button, a keyboard, a mouse, and the like. In addition, the operation unit 35 may include a speech recognition unit which recognizes an operation instruction by the user's speech, a biological authentication unit which authenticates a biological part of the user, or an image recognition unit which recognizes the user's facial expression or gesture.

The sensor I/F 36 is a communication interface for communicating with the measuring terminal 2 and sensor 3. The sensor I/F 36 receives data from the measuring terminal 2 and sensor 3, and transmits operation instructions to the measuring terminal 2 and sensor 3. In addition, the sensor I/F 36 may include an interface for the measuring terminal 2, and an interface for the sensor 3. The communication by the sensor I/F 36 may be wireless communication or wire communication.

In the present embodiment, the description is given on the assumption of a mode in which the sensor I/F 36 communicates with the measuring terminal 2 and sensor 3 by short-range wireless communication. However, aside from this, the sensor I/F 36 may include an interface for communicating with the measuring terminal 2 or sensor 3 via a communication cable. In addition, the sensor I/F 36 may execute serial communication via a communication cable, or may execute communication via a network such as a LAN.

Note that the sensor 3 may supply to the sensor I/F 36 measurement data in which a sensed signal is associated with time instant information, or may supply a sensed signal as measurement data to the sensor I/F 36. In the latter case, the controller 31 of the user terminal 11 may acquire measurement data in which data acquired from the sensor 3 is correlated with time instant information by the sensor I/F 36.

In the present embodiment, it is assumed that the sensor 3 is a sensor which measures SPO2. SPO2 is also used as an index indicative of a respiratory state of a human, and serves as an index indicative of the state of sleep apnea syndrome (SAS) of a human that is sleeping. Accordingly, when the user who is sleeping wears the sensor 3, the sensor 3 outputs measurement data of SPO2 which indicates the state of SAS of the user.

Next, a configuration of the server 12 will be described.

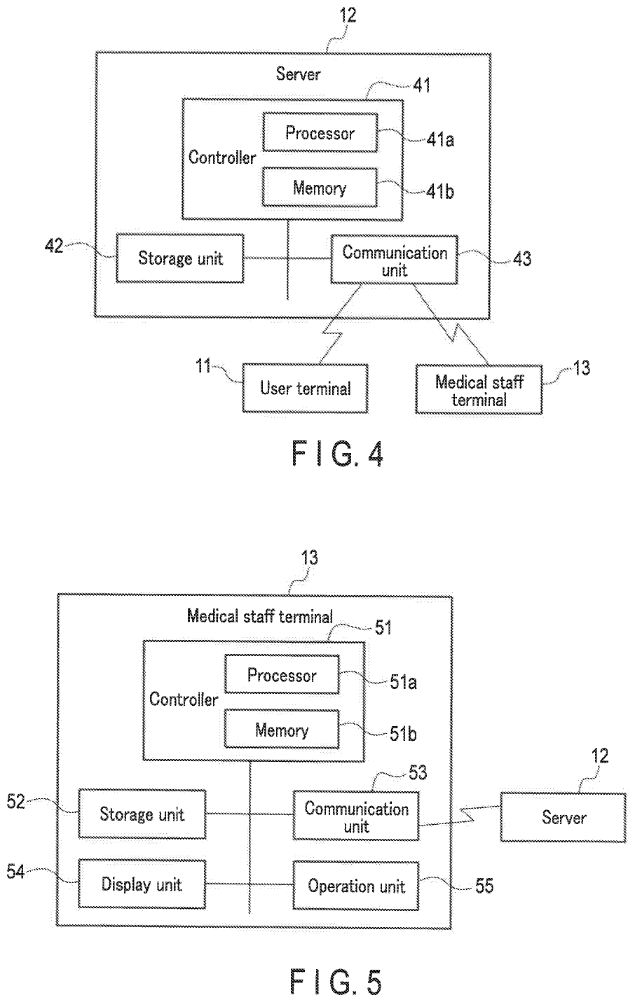

FIG. 4 is a block diagram illustrating a configuration example of the server 12 illustrated in FIG. 1.

The server 12 includes a controller 41, a storage unit 42 and a communication unit 43. In the present embodiment, the description is given on the assumption that a program (software) is installed in the server 12 so as to cause a general-purpose computer apparatus to execute process to be described later.

The controller 41 includes at least one processor 41a, and a memory 41b. The processor 41a executes programs by using the memory 41b, and thereby the controller 41 executes various kinds of operation control and data processing. The processor 41a is, for example, a CPU or an MPU including arithmetic circuitry. The memory 41b includes a nonvolatile memory which stores programs that the processor 41a executes, and a volatile memory such as a RAM which is used as a work memory. In addition, the controller 41 includes a clock (not shown) and includes a clock function which measures the present date/time.

The storage unit 42 is a data memory. The storage unit 42 is composed of, for example, a magnetic disk (HD), a semiconductor memory (a memory card, an SSD), an optical disc, a magneto-optical disc, or the like. The storage unit 42 stores various kinds of measurement data acquired from the user terminal 11. In addition, the storage unit 42 may store programs which the processor 41a of the controller 41 executes.

The communication unit 43 is a communication interface for communicating with the user terminal 11 or medical staff terminal 13. The communication unit 43 transmits data to the user terminal 11 or medical staff terminal 13 via a network, and receives data from the user terminal 11 or medical staff terminal 13. The communication by the communication unit 43 may be either wireless communication or wire communication. In the present embodiment, the description is given on the assumption of a configuration in which the communication unit 43 communicates with the user terminal 11 via a network such as the Internet, and communicates with the medical staff terminal 13 via a LAN. However, the communication by the communication unit 43 is not limited to a specific communication method.

Next, a configuration of the medical staff terminal 13 will be described.

FIG. 5 is a block diagram illustrating a configuration example of the medical staff terminal 13 illustrated in FIG. 1.

In the configuration example illustrated in FIG. 5, the medical staff terminal 13 includes a controller 51, a storage unit 52, a communication unit 53, a display unit 54 and an operation unit 55. In the present embodiment, the description is given on the assumption that the medical staff terminal 13 is a PC in which application software (program) is installed so that a process to be described later can be executed. However, the medical staff terminal 13 may be, for example, a communication terminal such as a tablet PC or a smartphone.

The controller 51 includes at least one processor 51a, and a memory 51b. The processor 51a executes programs by using the memory 51b, and thereby the controller 51 executes various kinds of operation control and data processing. The processor 51a is, for example, a CPU or an MPU including arithmetic circuitry. The memory 51b includes a nonvolatile memory which stores programs that the processor 51a executes, and a volatile memory such as a RAM which is used as a work memory. In addition, the controller 51 includes a clock (not shown) and includes a clock function which measures the present date/time.

The storage unit 52 is a data memory. The storage unit 52 is composed of, for example, a magnetic disk, a semiconductor memory (a memory card, an SSD), an optical disc, a magneto-optical disc, or the like. The storage unit 52 may store programs which the processor 51a of the controller 51 executes.

The communication unit 53 is a communication interface for communicating with the server 12. The communication unit 53 transmits data to the server 12 and receives data from the server 12. The communication by the communication unit 53 may be either wireless communication or wire communication. In the present embodiment, the description is given on the assumption that the communication unit 53 communicates with the server 12 via another kind of network such as a LAN. However, aside from this, the communication may include serial communication using a communication cable.

The display unit 54 includes a display screen (for example, an LCD, an EL display or the like). In the display unit 54, the display content, which is displayed on the display screen, is controlled by the control of the controller 51.

The operation unit 55 sends to the controller 51 an operation signal corresponding to an operation by the user (e.g. the subject to be measured). The operation unit 55 is, for example, a touch panel provided on the display screen of the display unit 54. The operation unit 55 is not limited to the touch panel, and may be an operation button, a keyboard, a mouse, and the like. In addition, the operation unit 55 may include a speech recognition unit which recognizes an operation instruction by the user's speech, a biological authentication unit which authenticates a biological part of the user, or an image recognition unit which recognizes the user's facial expression or gesture.

Next, functions which the controller 41 of the server 12 realizes will be described.

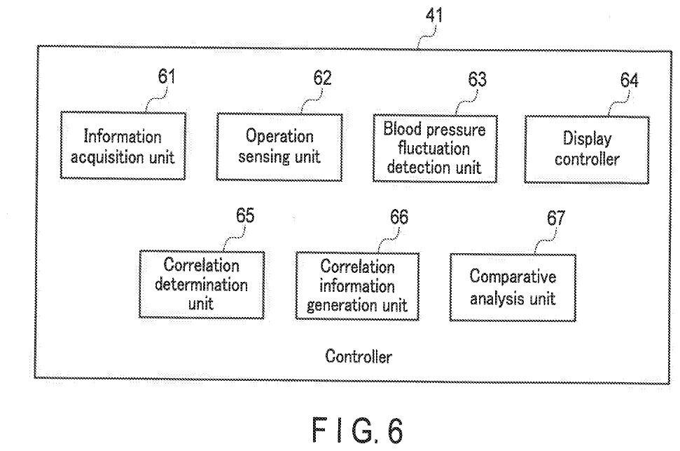

FIG. 6 is a block diagram illustrating functions which the controller 41 of the server 12 includes.

The controller 41 of the server 12 realizes various processing functions by the processor 41a executing programs stored in the memory 41b. As illustrated in FIG. 6, the controller 41 of the server 12 includes, as main functions, an information acquisition unit 61 (information acquisition circuit), an operation sensing unit 62, a blood pressure fluctuation detection unit 63 (blood pressure fluctuation detection circuit), a display controller 64, a correlation determination unit 65 (correlation determination circuit), a correlation information generation unit 66 (correlation information generation circuit), and a comparative analysis unit 67 (comparative analysis circuit).

The information acquisition unit 61 is a function of acquiring measurement data which the measuring terminal 2 and sensor 3 measure. The information acquisition unit 61 acquires, as the measurement data, successively measured blood pressure data and measurement data of a plurality of elements other than the blood pressure. As the measurement data of the plural elements, the information acquisition unit 61 acquires the measurement data of SPO2 which the sensor 3 measures, the acceleration data which the acceleration sensor 26 of the measuring terminal 2 measures, and the air temperature data which the air temperature sensor 28a of the measuring terminal 2 measures. The controller 41 executes, as the, information acquisition unit 61, a process of receiving various measurement data from the user terminal 11 via the communication unit 43, and storing the received measurement data in the storage unit 42. For example, the controller 41 may acquire measurement data which is transferred to the server 12 by a user's operation on the user terminal 11 or measuring terminal 2. In addition, the controller 41 may acquire measurement data from the user terminal 11 or measuring terminal 2, by requesting the user terminal 11 or measuring terminal 2 to transfer the measurement data.

The operation sensing unit 62 is a function of accepting an operation instruction from the user terminal 11 or medical staff terminal 13. The controller 41 executes, as the operation sensing unit 62, a process of receiving information indicative of the operation instruction from the user terminal 11 or medical staff terminal 13 via the communication unit 43, and accepting the received operation instruction.

The blood pressure fluctuation detection unit 63 is a function of detecting a blood pressure fluctuation of a reference value or more from the successively measured blood pressure data. The controller 41 executes, as the blood pressure fluctuation detection unit 63, a process of setting a reference value for a blood pressure fluctuation to be detected, and detecting a blood pressure fluctuation of the reference value or more, from the blood pressure data successively measured during a measurement period. In addition, the reference value for the blood pressure fluctuation may be set in multiple steps. In the present embodiment, for example, it is assumed that blood pressure surges of three-step risk degrees are detected as blood pressure fluctuations by three-step reference values.

Note that in the present embodiment, the description is given on the assumption that blood pressure surges of multiple-step risk degrees are detected as blood pressure fluctuations of reference values or more. However, a control ability to restore a raised blood pressure value a normal value may be detected as the blood pressure fluctuation.

In addition, the reference value for detecting a blood pressure surge is set, for example, for a magnitude of a blood pressure fluctuation and a period required for the blood pressure fluctuation. Specifically, when a large blood pressure fluctuation occurs in a short period, a blood pressure surge with a higher risk degree is determined. In addition, the reference value for the blood pressure surge may be set as a value corresponding to the activity amount of the user. For example, while the user is sleeping, it considered that the activity amount is substantially constant. On the other hand, while the user is up, it is also considered that the blood pressure fluctuation becomes large due to activity such as exercise. Thus, the blood pressure surge may be detected by taking into account not only the successively measured blood pressure data, but also measurement data of other elements such as the activity amount.

The display controller 64 is a function of controlling content that is to be displayed on the display unit of the medical staff terminal 13, user terminal 11 or measuring terminal 2. The controller 41 executes, as the display controller 64, a process of supplying display data, or data for display, to the medical staff terminal 13 or user terminal 11, which is communicable via the communication unit 43. The controller 41 executes, for example, display control of blood pressure-related information which is displayed on the display unit of the medical staff terminal 13, such that the successively measured blood pressure data relating to a specific user is associated with the measurement data of elements other than the blood pressure. The content displayed by the display controller 64 will be described later in detail.

The correlation determination unit 65 is a function of determining the correlation between a blood pressure fluctuation and measurement data of SPO2. The controller 41 executes, as the correlation determination unit 65, a process of determining the correlation between a blood pressure fluctuation of the reference value or more and measurement data of SPO2. The controller 41 determines the correlation with a blood pressure surge, based on the fluctuation of measurement data of SPO2 in a period in which the blood pressure surge occurred as the blood pressure fluctuation of the reference value or more and in periods before and after the period in which the blood pressure surge occurred. As the correlation with the blood pressure surge is more conspicuous, the controller 41 determines that the correlation (SAS sensitivity) between the state of SAS, which the measurement data of SPO2 indicates, and the blood pressure surge is higher.

However, in the determination of the correlation, the correlation is determined based on not only the magnitude of the fluctuation of the measurement data of SPO2 corresponding to the blood pressure surge, but also the tendency of the fluctuation. For example, if a blood pressure surge occurs even when the variation of the measurement data of SPO2 is small, the correlation determination unit 65 may determine that the sensitivity to SAS, which SPO2 indicates, is high. In addition, the correlation determination unit 65 may determine a tendency that a blood pressure surge easily occurs due to a sharp change of SPO2 regardless of the width of the fluctuation, or a tendency that a blood pressure surge easily occurs due to a large fluctuation of SPO2 regardless of the speed of the change.

The correlation information generation unit 66 is a function of generating correlation information (e.g. a correlation graph) which indicates the correlation between the blood pressure fluctuation of the reference value or more and the measurement data of SPO2. The controller 41 executes, as the correlation information generation unit 66, a process of generating correlation information which indicates the correlation between the blood pressure fluctuation of the reference value or more and the measurement data of elements other than the blood pressure. For example, in accordance with an instruction to display the correlation between SPO2 and a blood pressure surge, the controller 41 creates a correlation graph indicative of the correlation between the SPO2 and blood pressure surge, and causes, by the display controller 64, the display unit 54 of the medical staff terminal 13, or the like, to display the created correlation graph.

The comparative analysis unit 67 is a function of comparing past measurement data and present (latest) measurement data. For example, the controller 41 generates, as the comparative analysis unit 67, a comparative display screen which displays, by comparison, the past measurement data and present measurement data, and causes, by the display controller 64, the display unit 54 of the medical staff terminal 13, or the like, to display the comparative display screen. In addition, the controller 41 also includes, as the comparative analysis unit 67, a function of comparing the past measurement data and present measurement data of the user, evaluating the effect of medical treatment, and providing guidance based on the evaluation result.

Next, the operation of the management system of measurement data, which is configured as described above, will be described.

The user (the subject to be measured) instructs, by operating the measuring terminal 2, measurement of various elements, including successive measurement of the blood pressure value. In accordance with the user's operation, the measuring terminal 2 executes successive measurement of various elements, and transfers measurement data, which is the measured result in the measurement period, to the server 12 via the user terminal 11. In addition, the user terminal 11 also acquires measurement data which the sensor 3 measures in a period including at least a part of the measurement period in the measuring terminal 2. The sensor 3 may execute measurement according to an instruction to start the measurement from the user terminal 11, or may execute measurement upon receiving an instruction to start the measurement from the measuring terminal 2 via the user terminal 11.

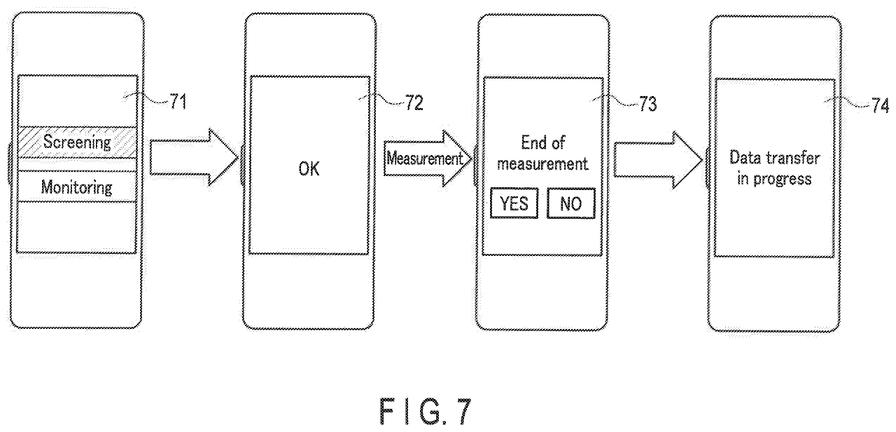

FIG. 7 is a view illustrating a transition example of an operation screen which the display unit 25 displays when the measuring terminal 2 executes measurement.

An operation screen 71 illustrated in FIG. 7 is a display example in a case of starting an operation mode of successive measurement (screening) in a measurement period. In the state in which the operation screen 71 is displayed, if the user touches “Screening” on the touch panel functioning as the operation unit 24, the controller 21 causes the display unit 25 to display an operation screen 72. The operation screen 72 is a confirmation screen for prompting the user to confirm the start of measurement in a measurement period that is set. Here, the measurement period may be a period from when the user instructs the start of measurement to when the user instructs the end of measurement, or may be a preset period. It is assumed that the measurement period is a period of sleep, a period of a specific activity, or a period of 24 hours. For example, when the period of sleep is set as the measurement period, the user instructs the start of measurement before sleep, and instructs the end of measurement after getting up.

If the user instructs the start of measurement on the operation screen 72 (i.e. instructs “OK”) , the controller 21 starts measurement. Upon starting the measurement, the controller 21 accumulates in the storage unit 23 the data which various sensors measure during the measurement period. Note that the controller 21 may transfer the measured data to the user terminal 11 at times as appropriate (in real time or at short cycles), and the user terminal 11 may accumulate the measurement data. In addition, before the instruction to start measurement is input, the controller 21 may enable the input of a measurement condition or the like by the user. For example, the controller 21 may set the measurement period which the user instructs before starting the measurement, or may accept personal identification information which the user instructs.

While the measurement is being performed, the controller 21 accepts an instruction to end the measurement by the user. When the user ends the measurement (for example, after getting up), the user causes the display unit 25 to display an operation screen 73 by a predetermined operation. For example, the controller 21 may cause the display unit 25 to display the operation screen 73 when sensing a predetermined motion on the measuring terminal 2 by the acceleration sensor 26, or may cause the display unit 25 to display the operation screen 73 when sensing a predetermined operation on the operation unit 24. Besides, after a preset measurement period has passed, the controller 21 may notify, by an alarm or the like, that the measurement period has passed, and may cause the display unit 25 to display the operation screen 73 which accepts the instruction to end the measurement.

The operation screen 73 displays a “YES” key and “NO” key as an instruction screen of the end of measurement. When the “YES” key is instructed in the operation screen 73, the controller 21 judges that the measurement is to be finished. When the “NO” key is instructed in the operation screen 73, the controller 21 judges that the measurement is to be continued.

When the controller 21 has judged that the measurement is to be finished, the controller 21 causes the display unit 25 to display an operation screen 74 which indicates guidance of transfer of measurement data. The operation screen 74 gives guidance indicating that the measurement data as the result of measurement during the measurement period is being transferred to the user terminal 11 or server 12. Here, it is assumed that the controller 21 stores in the storage unit 23 the measurement data measured during the measurement period (the measurement data in which the measurement values by the sensors are associated with the time instant information).

The controller 21 displays the operation screen 74 and transfers the measurement data during the measurement period, which is accumulated in the storage unit 23, to the server 12 via the user terminal 11 (or not via the user terminal 11) together with the personal identification information of the subject to be measured (or the measuring terminal 2). Thereby, the server 12 can acquire the measurement data of each user from each measuring terminal 2 in the management system of measurement data. In addition, the server 12 can collect measurement data of various elements of the user in the measurement period, by acquiring via the user terminal 11 the measurement data which the sensor 3 measures, together with the measurement data from the measuring terminal 2.

In the above-described example, it is assumed that, after the end of measurement, the measurement data during the measurement period is transferred to the server 12 via the user terminal 11. However, the measuring terminal 2 may transfer the measurement data to the user terminal 11 or server 12 in real time (or at predetermined cycles). If the frequency of communication is decreased, the measuring terminal 2 can hold down power consumption. On the other hand, if the measurement data is transferred from the measuring terminal 2 in real time or at predetermined cycles, the user terminal 11 or server 12 can acquire measurement data in real time or at predetermined cycles, and can analyze the state of the user in real time or at predetermined cycles.

Next, a description will be given of display examples of blood pressure-related information based on measurement data including blood pressure data which the server 12 successively measures.

Here, it is assumed that the server 12 causes the display unit 54 of the medical staff terminal 13 to display the information based on the measurement data which the measuring terminal 2 and sensor 3 measure. The display controller 64 of the server 12 causes the display unit 54 to display the information (blood pressure-related information) including the measurement data including successively measured blood pressure data and an analysis result based on the measurement data. The information, which the server 12 causes the display unit 54 to display, is information for supporting the determination by medical staff of the factor in the blood pressure surge, which is based on the measurement data, and the medical treatment advice by medical staff. However, the display controller 64 of the server 12 may cause the display unit 34 of the user terminal 11 or the display unit 25 of the measuring terminal 2 to display the blood pressure-related information such as the measurement data and the analysis result based on the measurement data.

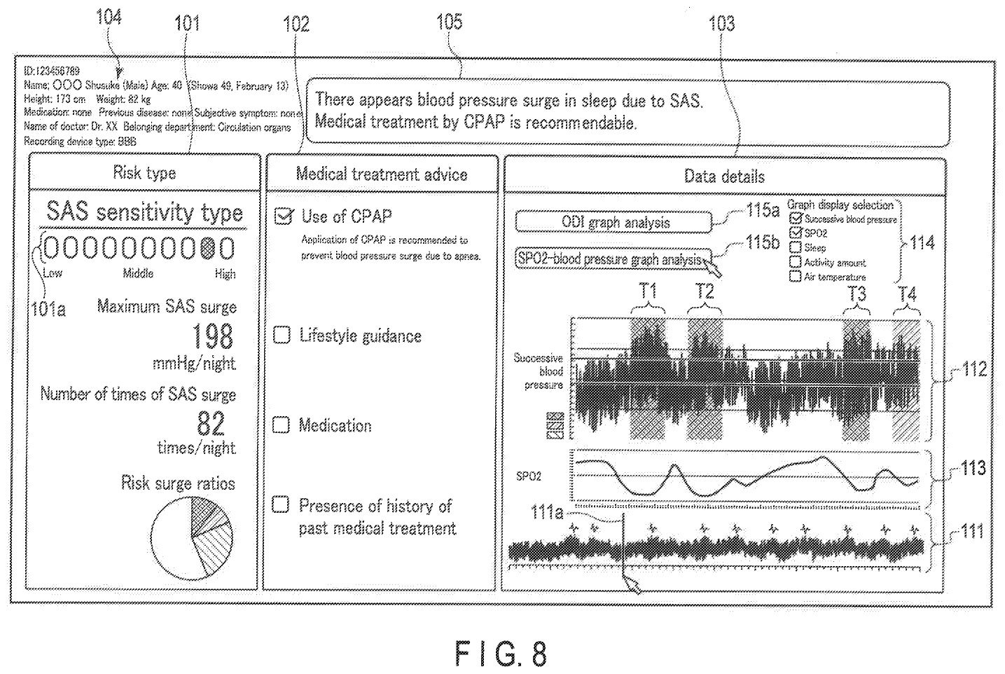

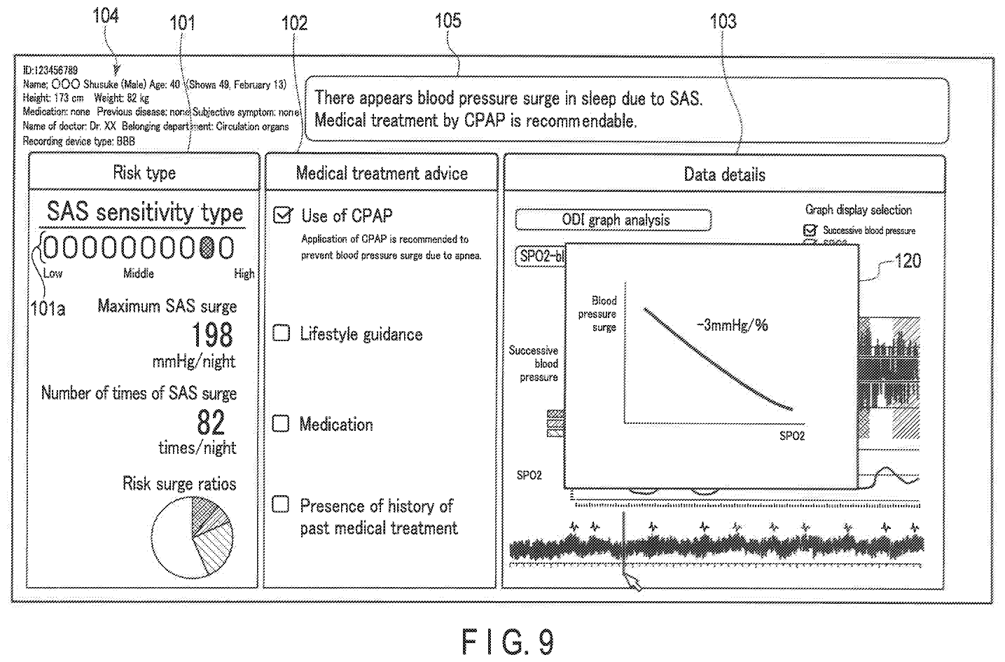

FIG. 8 and FIG. 9 are views illustrating display examples of blood pressure-related information, which the display controller 64 of the server 12 causes the display unit 54 of the medical staff terminal 13 to display.

FIG. 8 illustrates a display example of blood pressure-related information including measurement data in a specific measurement period of a certain user, and an analysis result relating to a blood pressure surge based on the measurement data.

A display screen illustrated in FIG. 8 includes a risk display area 101, an advice display area 102 and a data details display area 103. In addition, the display screen illustrated in FIG. 8 displays physical personal information 104 relating to the user (the subject to be measured) and a comment display field 105 which displays a comment such as a comprehensive determination result relating to a blood pressure surge which is indicated in the measurement result.

The risk display area 101 is an area which displays information indicative of the correlation (sensitivity) of SAS with a blood pressure surge, which the correlation determination unit 65 of the server 12 determines. In the example illustrated in FIG. 8, the risk display area 101 displays a determination result of SAS (sleep apnea syndrome) sensitivity, a maximum SAS surge, the number of times of SAS surge, and a circle graph indicative of the ratios of blood pressure surges according to risk degrees. For example, the circle graph, which is indicative of the ratios of blood pressure surges according to risk degrees, displays blood pressure surges, which are detected from the successively measured blood pressure data, by color-coding the blood pressure surges according to risk degrees. According to this circle graph, the ratio of risky blood pressure surges can be recognized at a glance.

It is assumed that the risk means a risk of the appearance of a cerebral cardiovascular event (cerebral infarction, cerebral hemorrhage, myocardial infarction, heart failure, or the like). In the present embodiment, the description is given on the assumption that the risk is determined by comprehensively considering elements such as a magnitude of a blood pressure fluctuation, a fluctuation time, and a change in fluctuation. For example, even if a difference in fluctuation is the same, it is considered that the risk is different between the case where the blood pressure fluctuates in several seconds and the case where the blood pressure fluctuates over a long time.

It should be noted, however, that the risk of the appearance of the cerebral cardiovascular event is determined based on various elements such as arterial sclerosis, gender, age, dietary contents, sleep and heredity, aside from the magnitude of the blood pressure fluctuation, fluctuation time, and change in fluctuation. Thus, the determination of the risk is not limited to the examples in the present embodiment, and the risk may be determined by taking into account various elements such as arterial sclerosis, gender, age, dietary contents, sleep and heredity.

In addition, the risk display area 101 displays a gauge 101a which indicates a degree of possibility of the occurrence of a blood pressure surge by SAS. The gauge 101a indicates a risk degree by a blood pressure surge in which SAS is a factor. The gauge 101a is not limited to the mode of display illustrated in FIG. 8. The gauge 101a may be a bar graph or a circle graph, or may display numerical values.

The advice display area 102 is an area which displays advice on medical treatment based on the state of occurrence of the blood pressure surge, and based on the correlation of SAS with the blood pressure surge. In the example illustrated in FIG. 8, as the treatment method for the blood pressure surge (i.e. the treatment method of SAS), a plurality of choices (“Use of CPAP”, “Lifestyle guidance” and “Medication”) are displayed or the advice display area 102, and information indicative of one treatment method, which is recommended from the choices, is displayed on the advice display area 102.

For example, the recommended treatment method may be selected based on the correlation between the blood pressure surge and the measurement data of SPO2 indicative of SAS. Specifically, the recommended treatment method may be selected in accordance with the risk degree of the blood pressure surge, in which SAS is assumed to be a factor. Besides, the treatment method may be selected in accordance with the frequency of occurrence of the blood pressure surge in which SAS is the factor.

In addition, the advice display area 102 displays information indicative of the presence/absence of a history of past medical treatment (history of diagnosis). When there is a history of past medical treatment, a comparison button 102a, which instructs comparison with past measurement data, is displayed. If the comparison button 102a is instructed, the display screen is updated to a comparison screen which compares past measurement data and present (latest) measurement data. The comparison screen with the past measurement data will be described later.

The data details display area 103 is an area which displays actual measurement data in detail. In the example illustrated in FIG. 8, the data details display area 103 displays, as the data details, a graph 111 of successively measured blood pressure data, a graph 112 in which a portion of the successively measured blood pressure data in a partial period is enlargedly displayed, a graph 113 in which measurement data of an element other than the blood pressure in the same period as in the graph 112 is enlargedly displayed, a select field 114 of grapy display, and analysis instruction buttons 115 (115a and 115b).

The graph 111 displays data of the blood pressure value successively measured during an entire measurement period in which successive measurement was conducted, or during a predetermined period (12 hours, 24 hours, or the like). The graph 111 displays an enlargement display slot 111a which indicates a period (enlargement period) for the enlarged display in the graphs 112 and 113.

The graph 112 displays blood pressure data in the period indicated by the enlargement display slot 111a of the graph 111. The graph 112 displays, by color coding, periods T1, T2, T3 and T4 which are detected as blood pressure surges.

The graph 113 displays measurement data of a selected element during the period indicated by the enlargement display slot 111a of the graph 111. The graph 113 is displayed by being associated with the graph 112. In the example illustrated in FIG. 8, the graph 113 is displayed such that the measurement data of SPO2 is associated with the blood pressure data of the graph 112.

The select field 114 of graph display indicates elements (indices) which are displayed as the graphs 112 and 113. In addition, the select field 114 is also used as a field for the medical staff or user to designate elements which are displayed as the graphs 112 and 113. The measurement data of the elements which are in the selected state in the select field 114 are displayed as the graph 112 and graph 113. In the example illustrated in FIG. 8, a successive blood pressure and SPO2 are in the selected state, and the graph 112 displays blood pressure data as the successive blood pressure, and the graph 113 displays the measurement data of SPO2. Items, which can be selected in the select field 114 at the same time, may be limited to items which can be displayed as the graphs 112 and 113. It is assumed that in the select field 114 in the initial state, blood pressure data and measurement data of an element, which is assumed to be a factor a blood pressure surge, are selected.

In addition, it should suffice if the elements that can be selected in the select field 114 are information which may become factors in the blood pressure fluctuation of the user, and are measurement data which the server 12 can acquire. In the example illustrated in FIG. 8, as well as the blood pressure, such elements as SPO2, sleep, activity amount and air temperature can be selected in the select field 114. In the above-described configuration example, the measurement data of SPO2 is measured by the sensor 3. The measurement data of sleep may be acceleration data (posture) which the acceleration sensor 26 of the measuring terminal 2 measures in connection with the subject to be measured, who is sleeping, or may be brain wave data which a brain wave sensor functioning as one of biological sensors 27 of the measuring terminal 2 measures. Further, the measurement data of the activity amount is acceleration data which the acceleration sensor 26 of the measuring terminal 2 measures. Besides, the measurement data of air temperature is measured by the temperature sensor 28a of the measuring terminal 2.

The analysis instruction buttons 115 are buttons for instructing specific analysis. In the example illustrated in FIG. 8, an analysis instruction button 115a, which instructs ODI analysis, and an analysis instruction button 115b, which indicates correlation of the blood pressure surge with SPO2, are displayed. The analysis instruction button 115b is a button which instructs display of correlation information (correlation graph) indicative of the correlation between measurement data of SPO2 and the blood pressure surge.

FIG. 9 is a view illustrating a display example of a correlation graph indicative of a correlation between SPO2 and a blood pressure surge. Specifically, when the analysis instruction button 115b illustrated in FIG. 8 is instructed, the controller 41 of the server 12 generates, by the correlation information generation unit 66, a correlation graph between SPO2 and the blood pressure surge, and causes the display unit 54 to display the generated correlation graph. In the display example illustrated in FIG. 9, a correlation graph between SPO2 and the blood pressure surge is displayed as a popup screen 120 on the display screen illustrated in FIG. 8. It is assumed that the correlation graph shown in FIG. 9 can be restored to the display state illustrated in FIG. 8 by an operator's operation.

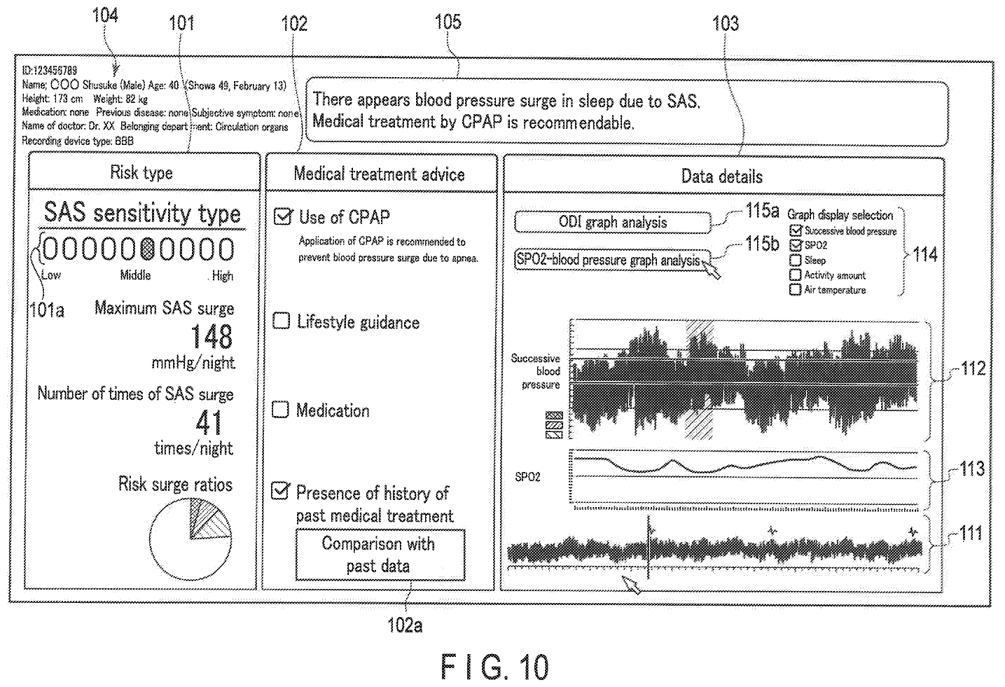

In addition, FIG. 10 illustrates a display example in which the advice display area 102 displays the presence of the history of past medical treatment and the comparison button 102a which instructs comparison with the past measurement data. If the comparison button 102a is instructed, the display screen is updated to a comparison screen which compares the past measurement data and present (latest) measurement data.

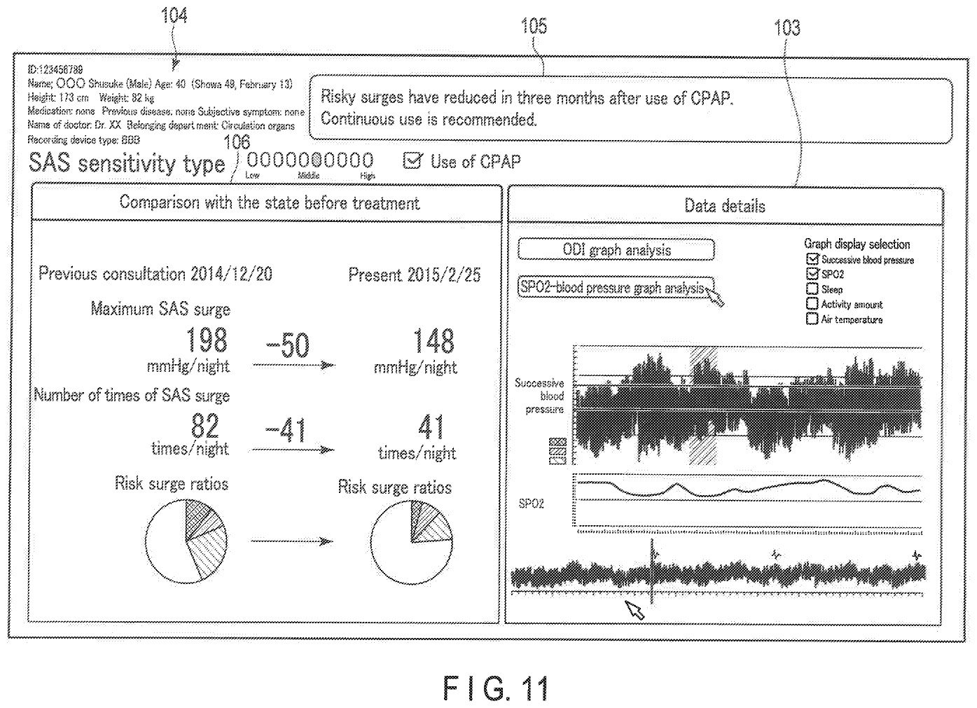

FIG. 11 is a view illustrating a display example of the comparison screen with the past measurement data.

In the example illustrated in FIG. 11, the “Maximum SAS surge”, “Number of times of SAS surge” and “Circle graph indicative of ratios of blood pressure surges according to risk degrees” are displayed by being associated with each of the past measurement data and the present measurement data. In addition, on the comparison screen illustrated in FIG. 11, the comment display field 105 displays an evaluation of the medical treatment effect obtained from the comparison result between the past measurement data and present measurement data, advice on a medical treatment method in the future, and the like.

The content (advice) displayed in the comment display field 105 is determined in accordance with the result of evaluation in multiple levels of the medical treatment effect by the controller 41, and the like. For example, the advice that is displayed may be a proposal to continue a previously proposed medical treatment method when an improvement by the medical treatment is recognized. On the other hand, when no improvement by the medical treatment is recognized, the advice that is displayed may be a proposal of a medical treatment method which is different from the previously proposed medical treatment method. Further, when a risky blood pressure surge is not detected, or when a detected blood pressure surge is within an allowable range in which the detected blood pressure surge can be diagnosed as being normal, the advice that is displayed may be a proposal to end the medical treatment or a proposal to conduct only general guidance on a lifestyle.

According to the display as described above, the server 12 can present the state of occurrence of a blood pressure surge, measurement data of SPO2 associated with blood pressure data, the possibility of occurrence of a blood pressure surge by SAS, and advance on medical treatment, and the like, to the medical staff or the user in an easy-to-understand manner. In addition, when there is past measurement data, the result of the medical treatment can be confirmed at a glance by the comparison screen which displays, by comparison, the past measurement data and present measurement data.

Next, a description will be given of a process of providing measurement data which the measuring terminal 2 and sensor 3 measure.

Here, an operation example is described in which the server 12 collects measurement data which the measuring terminal 2 and sensor 3 measure, and causes the medical staff terminal 13 to display the collected measurement data. However, such an operation may be implemented that a process, which the medical staff terminal 13 executes as will be described later, is replaced with a process which the user terminal 11 executes. In addition, a part or all of a process of the server 12, which will be described later, may be implemented by the user terminal 11 or measuring terminal 2.

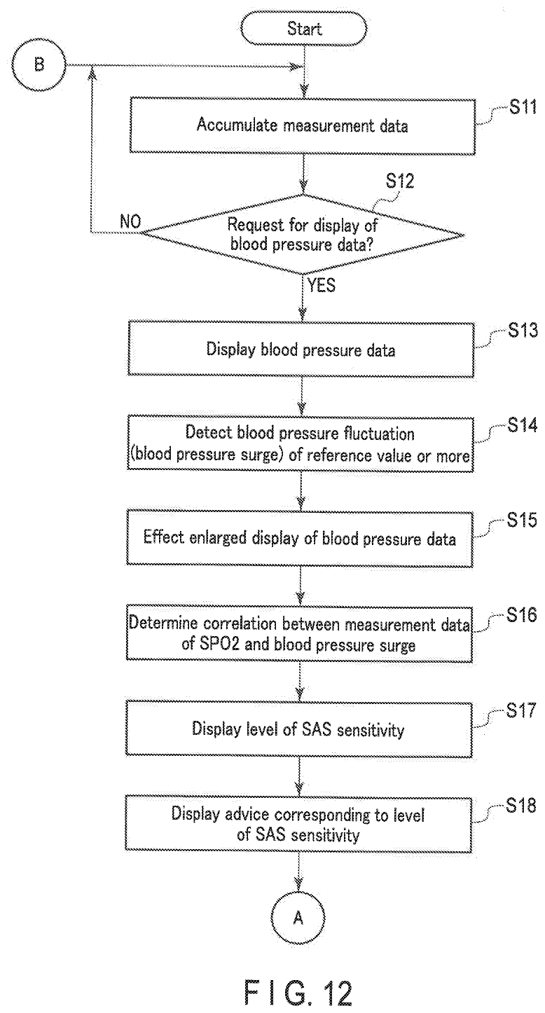

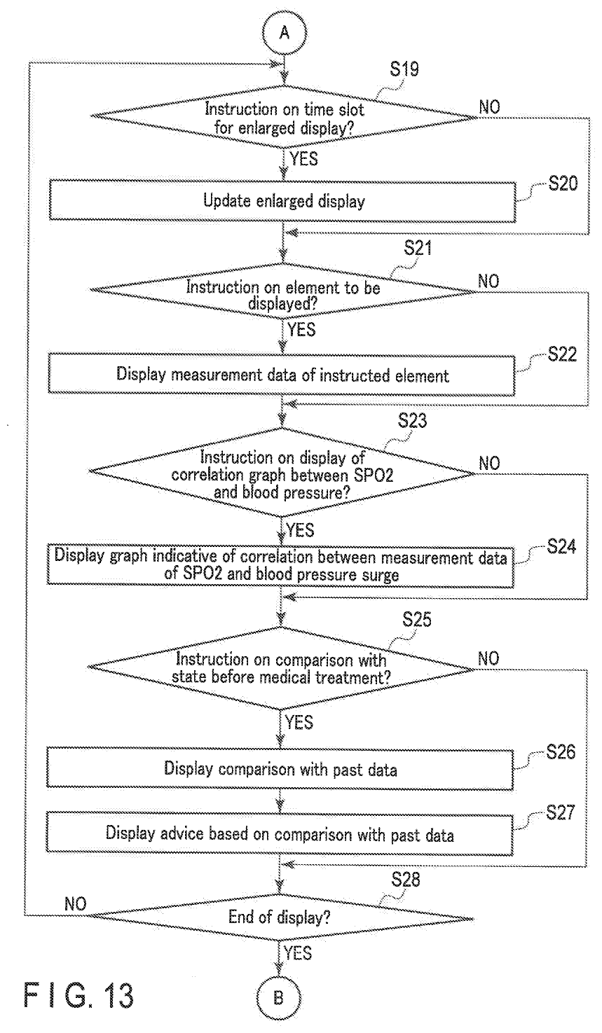

FIG. 12 and FIG. 13 are flowcharts for explaining an operation example of the server 12 functioning as the information processing apparatus according to the present embodiment.

When blood pressure-related information of a specific user is displayed on the display unit 54 of the medical terminal 13, the controller 51 of the medical staff terminal 13 transmits via the communication unit 53 a signal for requesting display information of blood pressure-related information from the server 12. For example, medical staff operates the operation unit 55 of the medical staff terminal 13 and instructs display of blood pressure-related information together with information for specifying the user (e.g. identification information of the user). If the display of the blood pressure-related information is instructed, the controller 51 of the medical staff terminal 13 transmits a signal for requesting, from the server 12, the display information of the blood pressure-related information together with the identification information of the user. At this time, the year/month/day (measurement period), in which the blood pressure-related information to be displayed was measured, may be designated, or items to be displayed may be preset.

The controller 41 of the server 12 acquires, by the information acquisition unit 61, measurement data from the measuring terminal 2 and sensor 3 via the user terminal 11, and accumulates the measurement data in the storage unit 42 (S11). The measurement data, which the information acquisition unit 61 acquires, includes measurement data of a blood pressure value which the measuring terminal 2 successively measures, and measurement data of SPO2 which the sensor 3 senses. In addition, the controller 41 executes a process of receiving identification information of the user together with the measurement data from the user terminal 11, and accumulating in the storage unit 42 the measurement data and the identification information of the user by correlating the measurement data and the identification information of the user.

In addition, while executing the process of accumulating the measurement data in the storage unit 42, the controller 41 accepts a display instruction of blood pressure-related information from the medical staff terminal 13. Upon receiving via the communication unit 43 a signal which requests the display of the blood pressure-related information (S12, YES), the controller 41 reads out the successively measured blood pressure data (blood pressure data during the measurement period of successive measurement, or blood pressure data successively measured in a predetermined period) corresponding to a designated user from the storage unit 42.

Upon reading out the successively measured blood pressure data of the designated user from the storage unit 42, the controller 41 causes, by the display controller 64, the display unit 54 of the medical staff terminal 13 to display the read-out blood pressure data (S13). For example, the controller 41 causes the display unit 54 to display the read-out successively measured blood pressure data as the graph 111 on the display screen as illustrated in FIG. 8.

In addition, the controller 41 detects, by the blood pressure fluctuation detection unit 63, a blood pressure fluctuation of a reference value or more in the read-out successively measured blood pressure data (S14). Here, it is assumed that blood pressure fluctuations of multiple-step reference values or more are detected as blood pressure surges according to risk degrees from the successively measured blood pressure data If the controller 41 detects blood pressure surges, the controller 41 determines a time slot (enlargement period) for enlargedly displaying the successively measured blood pressure data, based on the detection result of the blood pressure surges, and executes enlargement display of the blood pressure data in the determined enlargement period (S15). For example, the controller 41 causes the display unit 54 to display the blood pressure data in the enlargement period as the graph 112 on the display screen as illustrated in FIG. 8. In addition, with respect to the blood pressure data in the enlargement period that is displayed as the graph 112, the controller 41 causes the display unit 54 to display the time slots of blood pressure surges detected according to risk degrees, by color-coding the time slots. Thereby, the fluctuations of the blood pressure value in the enlargement period can be easily visually recognized, and the detected blood pressure surges can be easily recognized.

In addition, the controller 41 determines, by the correlation determination unit 65, a degree of possibility of the occurrence of a blood pressure surge by SAS, based on the correlation between the detected blood pressure surges and the measurement data of SPO2 (S16). For example, the controller 41 determines that the possibility of occurrence of the blood pressure surge by SAS is higher as the value of SPO2 is lower in the period in which the blood pressure surge occurred. In addition, the controller 41 determines that the possibility of the occurrence of the blood pressure surge by SAS is high, when the blood pressure surge occurs in accordance with a sharp result of the value of SPO2.

Upon determining the degree of possibility of the occurrence of the blood pressure surge by SAS, the controller 41 causes the display unit 54 to display information (level) indicative of the determined degree of possibility of the occurrence of the blood pressure surge by SAS (S17). In addition, the controller 41 determines advice on medical treatment corresponding to the degree of possibility of the occurrence of the blood pressure surge by SAS (S18). For example, the controller 41 selects, from the choices displayed in the advice display area of FIG. 8, a medical treatment method corresponding to the degree of possibility of the occurrence of the blood pressure surge by SAS, and causes the display unit 54 to display the medical treatment method. Besides, the controller 41 may create a comment corresponding to the degree of possibility of the occurrence of the blood pressure surge by SAS, and may display the comment in the comment display field 105.

By the above process, the server 12 causes the display unit 54 of the medical staff terminal 13 to display the display screen as illustrated in FIG. 8. When this display screen is displayed, the server 12 detects, by the operation detection unit 62, an operation by the operation unit 55 of the medical staff terminal 13, and updates the display content which the display unit 54 displays, in accordance with the operation instruction.

For example, the controller 41 of the server 12 accepts an instruction on the time slot (enlargement period) for the enlargement display as the graphs 112 and 113 (S19). For example, the controller 41 accepts the instruction on the enlargement period according to a position instructed on the graph 111 of the display screen illustrated in FIG. 8. If the enlargement period is designated by the operation unit 55 of the medical staff terminal 13 (S19, YES), the controller 41 of the server 12 updates the enlargement display slot 111a and the graphs 112 and 113 in accordance with the instructed enlargement period. Specifically, the controller 41 displays the enlargement display slot 111a, which indicates the enlargement period, at a position instructed on the graph 111 (S20). Further, the controller 41 newly sets the period, which the enlargement display slot 111a displays, as the enlargement period, and displays the measurement data in the enlargement period (the successively measured blood pressure data and the measurement data of the selected element other than the blood pressure) as the graphs 112 and 113.

In addition, the controller 41 accepts a select instruction on an element that is displayed as the graph 113 (S21). For example, on the display screen illustrated in FIG. 8, the controller 41 accepts a select instruction on the element that is displayed as the graph 113, in accordance with an instruction in the select field 114. When a change of the element to be displayed is instructed by the operation unit 55 of the medical staff terminal 13 (S21, YES), the server 12 changes the element that is displayed as the graph 113 to the instructed element, and displays the measurement data of the instructed element (S22). Further, the controller 41 of the server 12 changes the display such that the element instructed in the select field 114 is set in the selected state.

Besides, the controller 41 accepts, by the analysis instruction button 115b, a display instruction of a correlation graph indicative of the correlation between measurement data of SPO2 and blood pressure surge (S23). For example, on the display screen illustrated in FIG. 8, if it is detected that the analysis instruction button 115b is instructed (S23, YES), the controller 41 of the server 12 creates, by the correlation information generation unit 66, a graph (correlation graph) indicative of the correlation between the measurement data of SPO2 and the blood pressure surge, and causes the display unit 54 to display the created correlation graph (S24).

In addition, when there is past measurement data of the displayed user (when there is a history of medical treatment), the controller 41 of the server 12 displays the comparison button 102a on the display screen (S25). Upon detecting input of an instruction to the comparison button 102a (S25, YES), the controller 41 causes the display unit 54 to display the comparison screen with the past data as illustrated in FIG. 11 (S26). Further, the controller 41 compares, as the comparative analysis unit 67, the past measurement data and present measurement data, creates advice based on the comparison result, and displays the created advice in the comment display field 105 (S27).

In addition, if the controller 41 of the server 12 accepts an operation for the end of display in the medical staff terminal 13 (S28, YES), the controller 41 ends the display of the information relating to the successively measured blood pressure data. If the display is ended, the controller 41 returns to S11, and collects measurement data and accepts a display instruction.

As described above, the server 12 functioning as the information processing apparatus acquires the successively measured blood pressure data of the subject to be measure, and the measurement data of SPO2, and causes the display unit to display the degree of possibility of the occurrence of the blood pressure surge by SAS, which is determined based on the correlation between the blood pressure surge and the measurement data of SPO2. Thereby, it is possible for a person to save the labor of comparing, by viewing, an enormous amount of successively measured blood pressure data and measurement data of SPO2, and judging the correlation between SAS and the blood pressure fluctuation, and it is possible to present the degree of possibility of the occurrence of the blood pressure surge by SAS in an easy-to-view state for persons. As a result, it is possible to support medical treatment and diagnosis, such as healthcare, for improving the health condition of the subject to be measured.

The present invention is not limited directly to the above-described embodiments. In practice, the structural elements can be modified and embodied without departing from the spirit of the invention. Various inventions can be made by properly combining the structural elements disclosed in the embodiments. For example, some structural elements may be omitted from all the structural elements disclosed in the embodiments. Furthermore, structural elements in different embodiments may properly be combined.

A part or the entirety of the above-described embodiment can also be described as in the supplementary note below, but is not limited to the supplementary note.

### Supplementary Note 1

An information processing apparatus including:

a memory; and

at least one processor which cooperates with the memory,

the processor being configured to:

acquire, from a specific subject to be measured, measurement data of a successively measured blood pressure value and measurement data of SPO2;

detect a blood pressure fluctuation of a reference value or more from the acquired measurement data of the successively measured blood pressure value;

determine a degree of possibility of occurrence of a blood pressure fluctuation by sleep apnea syndrome, based on a correlation between the detected blood pressure fluctuation of the reference value or more and the measurement data of the SPO2; and

cause a display device to display information indicative of the determined degree of the possibility of the occurrence of the blood pressure fluctuation by the sleep apnea syndrome.

1. An information processing apparatus comprising:
an information acquisition circuit configured to acquire, from a specific subject to be measured, measurement data of a successively measured blood pressure value and measurement data of SPO2;
a blood pressure fluctuation detection circuit configured to detect, from the measurement data of the successively measured blood pressure value acquired by the information acquisition circuit, a blood pressure fluctuation which means a blood pressure surge of a reference value or more, the reference value being set for a magnitude of the blood pressure fluctuation and a period required for the blood pressure fluctuation;
a correlation determination circuit configured to determine a degree of possibility of occurrence of a blood pressure fluctuation by sleep apnea syndrome, based on a correlation with the measurement data of the SPO2 in a period which the blood pressure fluctuation of the reference value or more, which the blood pressure fluctuation detection circuit detects, occurs, and in periods before and after the period; and
a display controller configured to cause a display device to display information indicative of the degree of the possibility of the occurrence of the blood pressure fluctuation by the sleep apnea syndrome, which the correlation determination circuit determines.

2. The information processing apparatus of claim 1, wherein the display controller is configured to cause the display device to further display advice on medical treatment corresponding to the degree of the possibility of the occurrence of the blood pressure fluctuation by the sleep apnea syndrome, which the correlation determination circuit determines.

3. The information processing apparatus of claim 1, further comprising a comparative analysis circuit configured to acquire, when an instruction for comparison with past measurement data is given, past measurement data relating to the subject to be measured, who is being displayed, and configured to generate a comparison screen which compares measurement data, which is being displayed, and the past measurement data,
wherein the display controller is configured to cause the display device to display the comparison screen which the comparative analysis circuit generates in accordance with the instruction for the comparison with the past measurement data.

4. The information processing apparatus of claim 3, wherein the comparative analysis circuit is configured to create a comment based on the comparison between the measurement data, which is being displayed, and the past measurement data, and
the display controller is configured to cause the display device to further display the comment which the comparative analysis circuit creates, together with the comparison screen.

5. The information processing apparatus of claim 1, further comprising:
a correlation information generation circuit configured to generate a correlation graph which indicates a correlation between the measurement data of the SPO2 acquired by the information acquisition circuit and the blood pressure fluctuation of the reference value or more, which the blood pressure fluctuation detection circuit detects,
wherein the display controller is configured to cause the display device to display the correlation graph which the correlation information generation circuit generates in accordance with a display instruction of an operator.

6. The information processing apparatus of claim 1, wherein the information acquisition circuit is configured to acquire successively measured blood pressure data by a blood pressure sensor of any one of a PTT method, a tonometry method, optical method, a radio wave method, and ultrasonic method.

7. A non-transitory computer-readable storage medium storing an information processing program for causing a computer to execute:
a function of acquiring, from a specific subject to be measured, measurement data of a successively measured blood pressure value and measurement data of SPO2;
a function of detecting, from the acquired measurement data of the successively measured blood pressure value, a blood pressure fluctuation which means a blood pressure surge of a reference value or more, the reference value being set for a magnitude of the blood pressure fluctuation and a period required for the blood pressure fluctuation;
a function of determining a degree of possibility of occurrence of a blood pressure fluctuation by sleep apnea syndrome, based on a correlation with the measurement data of the SPO2 in a period in which the blood pressure fluctuation of the reference value or more, which the blood pressure fluctuation detection circuit detects, occurs, and in periods before and after the period; and
a function of causing a display device to display information indicative of the determined degree of the possibility of the occurrence of the blood pressure fluctuation by the sleep apnea syndrome.
